(19) United States
(12) Patent Application Publication
(10) Pub. No.: US 2008/0287507 A1
(43) Pub. Date: Nov. 20, 2008

(54) Nicotine containing toiletry waters

(76) Inventors: John Hedenstrom, Ramlosa (SE); Fredrik Nicklasson, Bjarreo (SE)

Correspondence Address: Johnson & Johnson, 201 Tabor Rd., Morris Plains, NJ, 07950, US

(21) Appl. No.: 11/803,745

(22) Filed: May 16, 2007

(30) Foreign Application Priority Data
May 16, 2007 (SE) SE 0701179-4

(51) Int. Cl. A61K 31/44; A61P 17/00

(52) U.S. Cl. 514/343

(57) ABSTRACT

A pharmaceutical liquid formulation for delivering nicotine in any form to a subject by transdermal uptake for treating tobacco dependence and similar conditions, said formulation being a toiletry-water. Toiletry-waters encompass aftershaves, eaux-de-parfum, eaux-de-toilette, eaux-de-cologne, toilet-waters and similar products, all in the form of a lotion, a balm or a gel. Embodiments for certain consideration are aftershave lotions, aftershave balms and aftershave gels.

### TECHNICAL FIELD

This invention relates to nicotine-containing toiletry-waters for treating tobacco dependence and similar conditions. Toiletry-waters encompass aftershaves, eaux-de-parfum, eaux-de-toilette, eaux-de-cologne, toilet-waters and similar products in the form of lotion, balm or gel. Embodiments for certain consideration are aftershave lotions, aftershave balms and aftershave gels.

### BACKGROUND OF THE INVENTION

Reduction of tobacco dependence is a desirable goal. In recent years, with the recognition of the harmful effects of tobacco smoking, there have been numerous campaigns and programs by governmental agencies and various health groups and other interested organisations to disseminate information about the adverse health effects resulting from tobacco smoking. Moreover, and as a result of this recognition of the harmful effects, there have been many programs directed to attempts in reducing smoking incidence.

Nicotine is an organic compound and is the principal alkaloid of tobacco. Nicotine is the chief addictive ingredient in the tobacco used in cigarettes, cigars, snuff and the like. Nicotine is also an addictive drug, and smokers characteristically display a strong tendency to relapse after having successfully stopped smoking for a time. Nicotine is the world's second most used drug, after caffeine from coffee and tea.

The main problem with tobacco smoking is its enormous implications on health. It is estimated that smoking related diseases cause some 3-4 million deaths per year. According to Centers for Disease Control and Prevention, cigarette smoking among adults—United States, 1995, MMWR 1997; 46:1217-1220 around 500,000 persons in USA die each year as a result of tobacco use. In fact, excessive smoking is now recognised as one of the major health problems throughout the world. This grim consequence of tobacco smoking has urged many medical associations and health authorities to take very strong actions against the use of tobacco.

Even though tobacco smoking is decreasing in many developed countries today it is hard to see how the societies could get rid of the world's second most used drug. The incidence of smoking is still rising in many countries, especially in less developed countries.

The most advantageous thing a heavy smoker can do is to stop smoking completely or at least to his/her smoking. Experience shows, however, that most smokers find this extremely difficult since, mostly, tobacco smoking results in a dependence disorder or craving. The World Health Organization (“WHO”) has in its International Classification of Disorders a diagnosis called Tobacco Dependence. Others like the American Psychiatric Association call the addiction Nicotine Dependence. It is generally accepted that these difficulties to stop smoking result from the fact that those heavy smokers are dependent on nicotine. The most important risk factors related to health are, however, substances that are formed during the combustion of tobacco, such as tar products, carbon monoxide, aldehydes, and hydrocyanic acid.

Effects of Nicotine

Nicotine is an addictive poisonous alkaloid C₅H₄NC₄H₇NCH₃, derived from the tobacco plant. Nicotine is also used as an insecticide. Approximately 40 milligrams of nicotine as a single dose may kill an adult (Merck Index). The administration of nicotine (for example, in the form of smoking a cigarette, cigar or pipe) can give a pleasurable feeling to the smoker. However, smoking has health hazards and it is, therefore, desirable to formulate an alternative way of administering nicotine in a pleasurable and harmless manner that can be used to facilitate withdrawal from smoking and/or used as a replacement for smoking.

When smoking a cigarette, nicotine is quickly absorbed into the smoker's blood and reaches the brain within around ten seconds after inhalation. The quick uptake of nicotine gives the consumer a rapid satisfaction, or kick. The satisfaction usually lasts during the smoking time of the cigarette and for a period of time thereafter. The poisonous, toxic, carcinogenic, and addictive nature of smoking has provided strong motivation to develop methods, compositions and devices, which can be used to break the habit of smoking cigarettes.

Nicotine Replacement Products

One way to reduce smoking is to provide nicotine in a form or manner other than by smoking and some products have been developed to fulfil this need. Nicotine containing formulations are currently the dominating treatments for tobacco dependence.

The successes in achieving reduction in the incidence of smoking have been relatively poor using presently known products. The present state of the art involves both behavioural approaches and pharmacological approaches. More than 80% of the tobacco smokers who initially quit smoking after using some behavioural or pharmacological approach to singly reduce smoking incidence generally relapse and return to the habit of smoking at their former rate of smoking within about a one year's period of time.

As an aid for those who are willing to stop smoking there are several ways and forms of nicotine replacement products available on the market. Several methods and means have been described for diminishing the desire of a subject to use tobacco, which comprises the step of administering to the subject nicotine or a derivative thereof as described in e g U.S. Pat. No. 5,810,018 (oral nicotine-containing spray), U.S. Pat. No. 5,939,100 (nicotine-containing starch micro spheres) and U.S. Pat. No. 4,967,773 (nicotine-containing lozenge).

Nicotine-containing nose drops have been reported (Russell et al., British Medical Journal, Vol. 286, p. 683 (1983); Jarvis et al., Brit. J. of Addiction, Vol. 82, p. 983 (1987)). Nose drops, however, are difficult to administer and are not convenient for use at work or in other public situations. Ways of administrating nicotine by way of delivering directly into the nasal cavity by spraying is known from U.S. Pat. No. 4,579,858, DE 32 41 437 and WO/93 127 64. There may be local nasal irritation, however, with use of nasal nicotine formulations. The difficulty in administration also results in unpredictability of the dose of nicotine administered.

Also, inhaling devices resembling a cigarette are known for uptake of nicotine vapours as suggested in U.S. Pat. No. 5,167,242. Said means and methods address the problems associated with addiction to nicotine.

One successful product that is used as a smoking substitute and/or as a smoking cessation aid and which is based on nicotine is the chewing gum Nicorette®. This product was one of the first nicotine replacement forms that was approved by the Food and Drug Administration (FDA) and is still one of the most used nicotine replacement products. Nicorette® chewing gum has been on the market in about 60 countries for several years. In this chewing gum the nicotine is present in the form of a complex with an insoluble cation-exchanger (polacrilex) that is dispersed in a gum base. The nicotine is slowly released from the gum due to chewing and will reach similar plasma levels as when smoking a cigarette after about 30 minutes depending on the chewing technique, i e slow or active. Patents related to this product are e g U.S. Pat. No. 3,877,468, U.S. Pat. No. 3,901,248 and U.S. Pat. No. 3,845,217.

Transdermal administration of nicotine has been described as use of skin patches for (Rose, in Pharmacologic Treatment of Tobacco Dependence, (1986) pp. 158-166, Harvard Univ. Press). Nicotine-containing skin patches that are in wide use today can cause local irritation and the absorption of nicotine is slow and affected by cutaneous blood flow.

WO 2005/011643 discloses certain biliquid foams, which may comprise active pharmaceutical ingredients. In a laundry list of suitable such ingredients is mentioned nicotine. Said foams together with aqueous gels may form stable dispersions that may in turn constitute e g anti-cellulite creams or aftershaves. There is no mentioning whatsoever of any utility for nicotine in this kind of formulation. Further, the formulation examples pertain to foams devoid of any active and to foams comprising as only active ibuprofen and caffeine. There is no example on any formulation comprising nicotine.

U.S. Pat. No. 6,479,076 discloses a nicotine-containing composition for dermal application in the form of a gel, ointment, solution, suspension or film. The composition forms a film upon administration and need comprise an uncrosslinked, water-insoluble vinylpyrrolidone copolymer being copolymerizable with a hydrophobic comonomer. Formulations according to the present invention are not film-forming and do not have to comprise such copolymer.

Prior Art and Problems Thereof

None of the nicotine-containing means known to date provides any product, which may fully accomplish rapid and at the same time agreeable transdermal administration of nicotine. Such an objective may be achieved using a nicotine-containing toiletry-water, which adds e g the features of lifestyle-appealing discreetness and agreeable smell.

### SUMMARY OF THE INVENTION

In order to accomplish rapid transdermal delivery of an active compound the pharmaceutical formulation to be used as well as the site of applying the formulation, i e the skin, should be optimized.

In order to be rapidly penetratable the skin should inter alia have as thin a stratum corneum, i e the horny layer of the epidermis, as possible. This may be achieved by e g thorough cleaning or scrubbing of the skin. A more effective and convenient method is shaving. Newly shaven skin, such as the cheeks for men and legs or arms for women or men, may therefore be very rapidly penetrated by e g an active compound.

An optimized pharmaceutical transdermal dosage form for applying to skin treated as above is a toiletry-water. For persons who regularly shave their cheeks or other parts of their body a preferred choice of toiletry-water is an aftershave lotion, an aftershave balm or an aftershave gel, preferably to be applied directly after shaving. For persons who do not shave their body most types of a nicotine-containing toiletry-water, such as an eau-de-parfum, an eau-de-toilette, an eau-de-cologne or a toilet-water, and even an aftershave lotion, an aftershave balm or an aftershave gel, will still be useful, especially when applied on a thin and preferably newly cleaned skin.

The captioned means are e g useful for delivering nicotine to a person, especially with the purpose to treat tobacco dependence.

An object of the present invention is to provide an efficient and effective liquid formulation, preferably being an aftershave lotion, an aftershave balm, an aftershave gel, an eau-de-parfum, an eau-de-toilette, an eau-de-cologne or a toilet-water, as well as methods and systems for uptake of nicotine in a subject.

Thus, the present invention provides a method for delivering nicotine in any form to a subject comprising applying to a subject a nicotine-containing toiletry-water, preferably being an aftershave lotion, an aftershave balm, an aftershave gel, an eau-de-parfum, an eau-de-toilette, an eau-de-cologne or a toilet-water.

The present invention also provides a method for obtaining reduction of the urge to smoke or use tobacco containing material and/or for providing a sense of smoking satisfaction without smoking, comprising the steps of replacing at least partly the tobacco containing material with the above liquid formulation, administering to a subject said liquid formulation containing nicotine in any form to the skin of the subject and allowing the nicotine in any form of the liquid formulation to be released and to be transdermally absorbed by the subject.

Furthermore, the present invention provides a system for delivering nicotine in any form to a subject, comprising said liquid formulation and at least one other means for obtaining reduction of the urge to smoke or use of tobacco as well as a system for obtaining reduction of the urge to smoke or otherwise use tobacco and/or for providing a sense of smoking satisfaction without smoking, comprising a liquid formulation as described above and at least one other method for obtaining reduction of the urge to smoke or otherwise use tobacco. Said system may be a system wherein the at least other method is selected from the group consisting of administration through mouth sprays, nasal sprays, transdermal patches, inhaling devices, lozenges, tablets and parenteral methods, subcutaneous methods, and transmucousal methods; or use of tobacco.

Except for the rapid transdermal delivery of nicotine the present invention provides the following advantages:
very good compliance due to administration in connection with daily toiletry routines,
easy and convenient masking of the nicotine odour,
suitable for both men and women,
breaks the smoking habit using means having no medical association, but being associated with hygiene and pleasure,
acts as well as a toiletry water in such a product's normal way,
provides a lifestyle approach making the product more acceptable than many other dosage forms,
provides good stability of the nicotine,
is suitable for administration with an appealing dispensing device.

### DETAILED DESCRIPTION OF THE INVENTION

### DEFINITIONS

Compound

Compound is the term used in perfumery to describe a concentrated fragrance mixture before it is diluted to make a finished perfume product. The compound may be carried in e g an oil and water base.

Toiletry-Water

Toiletry-water, also called perfume solution, is a liquid toiletry product comprising a small or large amount of compound.

Aftershave

Aftershave is mainly a men's toiletry product. It comes in the form of a lotion, a balm, or a gel. After shaving, aftershave is applied for one or more of a few reasons: It makes the skin look smoother; it soothes sensitive skin; it closes the pores after shaving; and it serves as light cologne. The cologne though usually is not strong enough to interfere with the man's primary cologne. The compound content is thus fairly low.

Eau-de-Cologne
Eau-de-cologne, or just cologne, is a perfume solution with around 3% compound.

Toilet-Water
Toilet-water is a light perfume solution similar to eau-de-cologne.

Eau-de-Toilette
Eau-de-toilette is a perfume solution with 3-8% compound.

Eau-de-Parfum
Eau-de-parfum is a perfume solution with 10-15% compound.

The above products when formulated as lotions usually have a low viscosity. Anyhow, they may be thickened to become balms and gels whereby they keep their main characteristics.

Balm
Balm is a viscous lipophilic toiletry water.

Gel
Gel is a viscous hydrophilic toiletry water.

Useful Liquid Formulations

The liquid formulations according to the present invention encompass toiletry-waters, comprising aftershave lotions, aftershave balms, aftershave gels, eaux-de-parfum, eaux-de-toilette, eaux-de-cologne, toilet-waters and similar products. Embodiments for certain consideration are aftershave lotions, aftershave balms and aftershave gels. Balms are especially useful when a lipophilic character is desired. Gels are especially useful when a hydrophilic character is desired. The user makes his or her choice between a lotion, a balm and a gel in the same way as had the choice been between ordinary toiletry waters devoid of nicotine.

The Active Ingredient

According to the invention, the present liquid formulations comprise nicotine in any form (for example free base, salt or complex).

With nicotine it is intended to include nicotine, 3-(1-methyl-2-pyrrolidinyl)-pyridine, with its base form, including synthetic nicotine as well as nicotine extracts from tobacco plants, or parts thereof, such as the genus Nicotiana alone or in combination; or pharmaceutically acceptable salts.

In preferred embodiments, the nicotine in any form is selected from the group consisting of the free base form of nicotine, a nicotine salt, a nicotine derivative, a nicotine inclusion complex or nicotine in any non-covalent binding, and mixtures thereof.

Numerous nicotine salts are known, and may be used, e g the salts presented in Table 1, preferably monotartrate, hydrogen tartrate (also called bitartrate or bitartrate di-hydrate), citrate, malate, and/or hydrochloride.

TABLE 1
Possible acids used for nicotine salt formation

| Acid | Molar ratio* of acid:nicotine |
|---|---|
| Formic | 2:1 |
| Acetic | 3:1 |
| Propionic | 3:1 |
| Butyric | 3:1 |
| 2-Methylbutyric | 3:1 |
| 3-Methylbutyric | 3:1 |
| Valeric | 3:1 |
| Lauric | 3:1 |
| Palmitic | 3:1 |
| Tartaric | 2:1 |
| Citric | 2:1 |
| Malic | 2:1 |
| Oxalic | 2:1 |
| Benzoic | 1:1 |
| Gentisic | 1:1 |
| Gallic | 1:1 |
| Phenylacetic | 3:1 |
| Salicylic | 1:1 |
| Phthalic | 1:1 |
| Picric | 2:1 |
| Sulfosalicylic | 1:1 |
| Tannic | 1:5 |
| Pectic | 1:3 |
| Alginic | 1:2 |
| Hydrochloric | 2:1 |
| Chloroplatinic | 1:1 |
| Silicotungstic | 1:1 |
| Pyruvic | 2:1 |
| Glutamic | 1:1 |
| Aspartic | 1:1 |

*recommended at the time of production

The inclusion complex may comprise a cyclodextrin, such as β-cyclodextrin. Such complexes are especially useful in balms and gels.

One or more additives may be added to the present liquid formulation. Additives are further described in the below paragraph Other additives to the oral formulation.

Amount and Distribution of the Nicotine in the Liquid Formulation

The nicotine in any form according to the invention is formulated to provide the subject with a dose to achieve an effect. The effect may be to provide a sense of smoking satisfaction without smoking. Another effect of the administered nicotine in any form may be a reduction of the urge to smoke or use tobacco.

The effect may also be a combination of reduction of said urge and providing a sense of smoking satisfaction without smoking. The amount of the nicotine should be sufficient to provide such an effect in a subject. This amount may, of course, vary from person to person.

According to the invention, embodiments of the liquid formulation comprise embodiments wherein nicotine in any form is present in an amount of 0.05-15 mg calculated as the free base form of nicotine per unit dose of the liquid formulation. This may in different embodiments include 0.1, 0.5, 1, 2, 3, 4, 5, 6, 8, 10, 12 and 15 mg calculated as the free base form of nicotine per unit dose. Preferably the present formulation is dispensed with a metered dispensing device provided with child resistant safety measures.

The number of unit doses to be administered to a subject per 24 hours depends on how heavy a tobacco user the subject has been and on how far the subject has advanced in his process of weaning off from tobacco. Typically the number of unit doses per 24 hours is between 1 and 3. For a single dose per 24 hours up to 15 mg nicotine or more is a suitable dose, while up to 6 mg nicotine is a suitable dose if 3 doses per 24 hours are administered.

The nicotine in any form may be distributed in the liquid formulations in different embodiments. Different distributions of the nicotine throughout the liquid formulations will imply administration of the nicotine to the subject in different ways. This may, then, provide several possibilities to adjust the composition of the liquid formulation according to different needs of different subjects depending on the urge to smoke or use tobacco of the subject. In the below Examples are disclosed different such embodiments.

Other Additives to the Liquid Formulation

Other additives may be added optionally to the liquid formulation according to the knowledge of the skilled person.

Optional additives of certain interest are e g transdermal permeability enhancers for increasing the transdermal uptake of nicotine. Also of interest are fragrances used in the perfume industry. As nicotine itself has a very bad taste the product as such is non-appealing for ingestion by e g children. If needed one may though add bitter-tasting compounds for increasing the product's non-appealing taste.

Method for Delivering Nicotine in Any Form to a Subject

The invention may be used to deliver nicotine to the subject (person) in a variety of ways. According to one embodiment of the invention, a method for delivering nicotine in any form to a subject comprises the steps of:

a) administering, preferably under light rubbing, to a subject a liquid formulation containing nicotine in any form according to the invention onto the skin of the subject, and

b) allowing the nicotine in any form in the liquid formulation to be released and absorbed into the blood plasma of the subject.

Means for Administering the Liquid Formulation

The present liquid formulation may e g be administered using a roll-on type bottle, a dab bottle or a spray bottle. Preferably such administration means provides for metered dosing and child resistance features.

For convenience a larger administration device or container may be kept at home, while a smaller device may be brought along in a pocket or a handbag.

Method for Obtaining Reduction of the Urge to Smoke or Use of Tobacco

Another feature of the invention is the ability to use the invention to reduce the urge to smoke. A method for obtaining reduction of the urge to smoke or use tobacco containing material and/or for providing a sense of smoking satisfaction without smoking according to the invention comprises the steps of:

a) replacing at least partly the tobacco containing material with a liquid nicotine-containing formulation,

b) administering to a subject a liquid formulation containing nicotine in any form onto the skin of the subject, and

Further embodiments of the method for delivering nicotine to a subject may comprise the steps of combining administration of the liquid formulation with at least one other method for obtaining reduction of the urge to smoke or use of tobacco.

Sustained Reduction of the Urge to Smoke or Use of Tobacco

The invention may also be used to reduce the urge to smoke or use tobacco. Still, to continue the feeling or sense of satisfaction of the subject, and to avoid that the craving returns, a sustained craving relief may be obtained after the initial craving relief. A sustained craving relief is obtained by using the liquid formulation in such a way as to allow a sustained uptake of the nicotine. The sustained craving relief and/or feeling or sense of satisfaction of the subject will continue as long as the subject maintains the blood plasma levels of nicotine at a level high enough to reach this sense of feeling.

Cessation of the Urge to Smoke or Use of Tobacco

For some of the users, it may be a goal to terminate the usage of nicotine completely, due to several reasons e g health, economical, social or behavioural. This cessation of smoking or the urge to use tobacco may be achieved by further decreasing the amount of nicotine in any form gradually over time. In a specific embodiment of the invention, the method described above for obtaining craving relief may further comprise the steps of decreasing the amount of nicotine in the liquid formulation described above gradually over time, so as to achieve a complete relief of tobacco craving. This method results in a weaning process gradually over time.

Different types of smokers reach the sense of reduced craving at different plasma levels of nicotine. This may, of course, affect the individual types of administration programs of a liquid formulation according to the invention. Different types of smokers include e g peak seekers or smokers that crave for a plasma level of nicotine constantly being above the level for withdrawal symptoms.

One strategy may be to lower the frequency of the administered liquid formulation. Other embodiments include varying the dose of the nicotine in said liquid formulation as well as the combination of these two. Also, the strategy may include a liquid formulation with substantially no nicotine in any form. Such a liquid formulation may be administered at the end of the treatment period, when the craving is low or substantially absent.

Systems for Delivering Nicotine and for Obtaining Craving Relief

According to the invention there is a system for delivering nicotine in any form to a subject particularly for obtaining craving relief. Such a system comprises a liquid formulation according to the invention and at least one other means for obtaining reduction of the urge to smoke.

Another system according to the invention may also be a system for obtaining reduction of the urge to smoke or use of tobacco and/or for providing a sense of smoking satisfaction without smoking. Such a system comprises a liquid formulation according to the invention and at least one other method or means for obtaining reduction of the urge to smoke or use tobacco. Other methods and means may also be a concomitant or concurrent method selected from the group consisting of administration through mouth sprays, nasal sprays, transdermal patches, inhaling devices, lozenges, tablets and parenteral methods, subcutaneous methods, and transmucousal methods; or use of tobacco.

In a specific embodiment, the at least other method comprises administration of nicotine.

Use of the Liquid Formulation

The use of the liquid formulation according to the invention may include obtaining a fast and/or sustained and/or complete reduction of the urge to smoke and use tobacco or for providing a sense of smoking without smoking as described above.

The dose of nicotine is chosen to give the subject an individual sensory perception and satisfaction with an effect of the nicotine in any form. The use of a liquid formulation may also be a sole use according to the invention or a combination with other means or methods known in the field of drug abuse. Specifically, the present invention may be used in combination with other means as described above in the methods in the paragraphs above.

The use may give a quick reduction of the urge to smoke or use tobacco.

Other embodiments will imply a use giving a slow reduction of the urge to smoke or use tobacco.

Use for Therapy and Treatment

The liquid formulation according to the invention may be used in therapy and treatment. Said therapy may be a treatment of a disease selected from the group consisting of tobacco or nicotine dependence, Alzheimer's disease, Crohn's disease, Parkinson's disease, Tourette's syndrome, ulcerous colitis and post-smoking-cessation weight control.

Nicotine may also be used for a liquid formulation according to the invention for the treatment of said diseases.

Further, nicotine may be used in the production of a nicotine-containing liquid formulation according to the invention for the treatment of said diseases.

Production of the Liquid Formulation

The liquid formulations according to the present invention are basically produced according to methods known in the art. Exemplary, but not limiting, production methods are provided below under Examples.

Conveniently, the compositions of additives according to the invention, e g the buffer system, are made simultaneously, according to known procedures in the art for formulating e g the buffers. Depending on the physical properties of the buffer system incorporated, it may be convenient to add the buffer system/s either with the liquid part or with the solid part of the composition. In the case of buffering systems available as fine powders, it may, of course, be most convenient to add those powders with the solid, powdered part of other additives.

The final product may then be analysed and further wrapped.

Analysis of Nicotine

The analysis of nicotine uptake and effect according to the invention may be done according to standard procedures known in the art, e g using bioanalysis for the determination of nicotine or its metabolites in the plasma of a subject.

### EXAMPLES

The below examples on embodiments of the present invention are illustrative and non-limiting. The skilled person may on the basis of the following examples envisage also other embodiments of the present invention. Batch sizes for the manufacture of the below formulations may be modified according to the actual need and to the actual production facilities. If not stated otherwise procedures and equipment known in the art are used in the below manufacturing.

### Example 1

### Aftershave Lotion

| Ingredient | Function | Amount per application | |
|---|---|---|---|
| Nicotine free base | Active ingredient | 10 | mg |
| Water, purified | Solvent | About 55 | mg |
| Ethanol | Adstringent/Solvent | 35 | mg |

| | | |
|---|---|---|
| Perfume | Fragrance | q.s |

| | | |
|---|---|---|
| Total: | 100 | mg |

### Manufacturing

1. Add ethanol to purified water at room temperature.

2. Add nicotine free base under stirring.

3. Add perfume under stirring.

### Example 2

### Aftershave Lotion

| | | |
|---|---|---|
| | | Amount per |
| Ingredient | Function | application |

A

| | | | |
|---|---|---|---|
| PEG-30 | Emulsifier | 2.00 | mg |
| Dipolyhydroxystearate | | | |
| Paraffin, Liquid | Oil phase | 7.00 | mg |
| Isohexadecane | Oil phase | 5.00 | mg |
| Caprylic/Capric Triglyceride | Oil phase | 7.00 | mg |
| Ethylhexyl Palmitate | Oil phase | 8.00 | mg |
| PPG-15-Stearyl Ether | Emulsifier | 3.50 | mg |
| Castor Wax | Oil phase | 0.80 | mg |
| Microcrystalline Wax | Oil phase | 1.20 | mg |
| Cyclomethicone | Emollient | 1.47 | mg |
| Propylparahydroxybenzoate | Preservative | 0.03 | mg |

B

| | | | |
|---|---|---|---|
| Water, Purified | Aqueous solvent | About 58.9 | mg |
| Methylparahydroxybenzoate | Preservative | 0.10 | mg |

C

| | | | |
|---|---|---|---|
| Nicotine free base | Active pharmaceutical | 5.00 | mg |
| | ingredient | | |

| | | |
|---|---|---|
| Perfume | Fragrance | q.s. |

| | | |
|---|---|---|
| Total: | 100 | mg |

### Manufacturing

1. Heat A and B separately to 75° C.

2. Slowly add B to A under intensive stirring.

3. Cool to 40° C., maintaining vigorous stirring.

4. Add C under intensive stirring.

5. Cool to room temperature, maintaining vigorous stirring

### Example 3

### Aftershave Balm

| | | |
|---|---|---|
| | | Amount per |
| Ingredient | Function | application |

A

| | | | |
|---|---|---|---|
| Paraffin, Liquid | Oil phase | 7.50 | mg |
| Polyetylenglycol-5- | Emulsifier | 4.70 | mg |
| glycerylstearate | | | |
| Cetostearyl alcohol | Oil phase | 2.80 | mg |
| Stearic Acid | Oil phase | 2.37 | mg |
| Propylparahydroxybenzoate | Preservative | 0.03 | mg |

B

| | | | |
|---|---|---|---|
| Water, Purified | Aqueous solvent | about 77.5 | mg |
| Methylparahydroxybenzoate | Preservative | 0.10 | mg |

C

| | | | |
|---|---|---|---|
| Nicotine free base | Active pharmaceutical | 5.00 | mg |
| | ingredient | | |

| | | |
|---|---|---|
| Perfume | Fragrance | q.s. |

| | | |
|---|---|---|
| Total: | 100 | mg |

### Manufacturing

1. Heat A and B separately to 80° C.

2. Slowly add A to B while stirring.

3. Homogenize the mixture during 1 minute.

4. Allow to cool while stirring moderately.

5. Add C below 40° C. while stirring moderately.

6. Allow to cool to room temperature while stirring moderately.

### Example 4

### Aftershave Gel

| | | |
|---|---|---|
| | | Amount per |
| Ingredient | Function | application |

A

| | | | |
|---|---|---|---|
| Nicotine free base | Active pharmaceutical | 5.00 | mg |
| | ingredient | | |
| Water, Purified | Solvent | 75.2 | mg |
| Polyoxyethylene-9-lauryl | Emulsifier | 2.0 | mg |
| ether (Laureth-9) | | | |
| Chamomille Extract, Liquid | Anti-irritant, fragrance | 2.0 | mg |

B

| | | | |
|---|---|---|---|
| Polyoxyethylene sorbitol | Emulsifier | 10.0 | mg |
| (Sorbeth-30) | | | |
| Methylparahydroxybenzoate | Preservative | 0.10 | mg |

C

| | | | |
|---|---|---|---|
| Carbomer (Carbopol ETD | Gelling agent | 0.5 | mg |
| 2001) | | | |

D

| | | | |
|---|---|---|---|
| Ethanol | Solvent | 5.0 | mg |
| Menthol | Cooling agent, | 0.2 | mg |
| | fragrance | | |

E

| | | |
|---|---|---|
| Triethanolamine | Buffering agent | To pH 6.0-6.5 |

| | | |
|---|---|---|
| Total: | 100 | mg |

### Manufacture

1. Mix A at room temperature.

2. Add B successively while stirring.

3. Slowly add C and stir until completely dissolved.

4. Add D while stirring.

5. Neutralize product by adding E and continue stirring for 15 minutes.

Lotions, balms and gels of eau-de-perfum, eau-de-cologne and toilet water may be manufactured by adapting the manufacturing methods of the above Examples.

1. A liquid formulation for administering nicotine comprising a toiletry-water containing nicotine in an amount effective for transdermal administration to a subject.

2. The liquid formulation according to claim 1 wherein said toiletry-water is selected from the group consisting of an aftershave, an eau-de-parfum, an eau-de-toilette, an eau-de-cologne or a toilet-water.

3. The liquid formulation according to claim 1 wherein said toiletry-water is in the form of a solution, a balm or a gel.

4. The liquid formulation according to claim 3 wherein said toiletry-water is an aftershave lotion, an aftershave balm or an aftershave gel.

5. The liquid formulation according to claim 1 wherein said nicotine is selected from the group consisting of a nicotine salt, the free base form of nicotine, a nicotine derivative, a nicotine inclusion complex or nicotine in any non-covalent binding; and mixtures thereof.

6. The liquid formulation according to claim 5 wherein said nicotine inclusion complex is a nicotine-cyclodextrin complex.

7. The liquid formulation according to claim 6 wherein said nicotine-cyclodextrin complex is nicotine-β-cyclodextrin.

8. The liquid formulation according to claim 5 wherein said nicotine salt is a mono-tartrate, hydrogen tartrate, citrate, malate and/or hydrochloride salt.

9. The liquid formulation according to claim 1 wherein said nicotine is present in an amount from about 0.05-15 mg calculated as the free base form of nicotine per unit dose.

10. The liquid formulation according to claim 9 wherein said nicotine is present in an amount from about 6-15 mg calculated as the free base form of nicotine per unit dose.

11. The liquid formulation according to claim 9 wherein said nicotine is present in an amount from about 0.05-6 mg calculated as the free base form of nicotine per unit.

12. The liquid formulation according to claim 1 further comprising one or more additives selected from the group consisting of transdermal penetration enhancers, compounds with bitter or other unpleasant taste, and mixtures thereof.

13. A method for delivering nicotine to a subject comprising the steps of
a) administering to a subject a liquid formulation according to claim 1 onto the skin of the subject, and
b) allowing the nicotine in said liquid formulation to be released and absorbed into the systemic circulation of the subject.

14. The method for delivering nicotine to a subject according to claim 13 wherein said liquid formulation is a toiletry-water is selected from the group consisting of an aftershave, an eau-de-parfum, an eau-de-toilette, an eau-de-cologne or a toilet-water.

15. The method for delivering nicotine to a subject according to claim 14 wherein said toiletry-water is in the form of a solution, a balm or a gel.

16. The method for delivering nicotine to a subject according to claim wherein 13 said nicotine is selected from the group consisting of a nicotine salt, the free base form of nicotine, a nicotine derivative, a nicotine inclusion complex or nicotine in any non-covalent binding; and mixtures thereof.

17. The method for delivering nicotine to a subject according to claim 16 wherein said nicotine inclusion complex is a nicotine-cyclodextrin complex.

18. The method for delivering nicotine to a subject according to claim 17 wherein said nicotine-cyclodextrin complex is nicotine-β-cyclodextrin.

19. A method for delivering nicotine to a subject according to claim 13 wherein said nicotine is present in an amount from about 0.05-15 mg calculated as the free base form of nicotine per unit dose.

20. A method for delivering nicotine to a subject according to claim 19 wherein said nicotine is present in an amount from about 6-15 mg calculated as the free base form of nicotine per unit dose.

21. A method for delivering nicotine to a subject according to claim 19 wherein said nicotine is present in an amount from about 0.05-6 mg calculated as the free base form of nicotine per unit dose.
